(19) United States
(12) Patent Application Publication
(10) Pub. No.: US 2006/0052356 A1
(43) Pub. Date: Mar. 9, 2006

(54) Pharmacological methods to modulate oxygen consumption

(76) Inventors: Kevin R. Ward, Glen Allen, VA (US); R. Wayne Barbee, Glen Allen, VA (US); Penny Reynolds, Gum Spring, VA (US); Robert Dieglemann, Richmond, VA (US); Rakesh Kukreja, Richmond, VA (US); Bruce Spiess, Manakin-Sabot, VA (US); Melissa Evans, Midlothian, VA (US); Eric Edwards, Richmond, VA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/952,525

(22) Filed: Sep. 29, 2004

Related U.S. Application Data
(60) Provisional application No. 60/607,071, filed on Sep. 3, 2004.

(51) Int. Cl. A61K 31/33

(52) U.S. Cl. 514/183

(57) ABSTRACT

Methods of modulating oxygen consumption in tissues are provided. In particular, the methods involve the administration of agents that transiently inhibit protein synthesis. The methods are used to treat ischemia and reperfusion-related injuries by temporarily decreasing oxygen consumption needs, and/or by inhibiting inflammation that results from reperfusion.

### CROSS-REFERENCE TO RELATED APPLICATIONS

This application claims benefit of U.S. provisional patent application 60/607,071 filed Sep. 3, 2004, the complete contents of which are hereby incorporated by reference.

### STATEMENT REGARDING FEDERALLY SPONSORED RESEARCH

This invention was made using funds from grants from the Department of Defense having grant number N66001-02-C-8052. The United States government may have certain rights in this invention.

### DESCRIPTION

### BACKGROUND OF THE INVENTION

1. Field of the Invention

The invention generally relates to methods of modulating oxygen consumption in an organism. In particular, the invention provides methods of transiently inhibiting protein synthesis and/or proteolysis in order to temporarily decrease oxygen consumption needs and to modulate the inflammatory response to tissue hypoxia and injury.

2. Background of the Invention

The cellular processes of protein synthesis and metabolism are very high energy consuming processes and require utilization of significant amounts of oxygen. When injury, illness or certain surgical procedures reduce oxygen delivery to tissues to critically low levels, (for example, during hemorrhagic and traumatic shock, cardiopulmonary bypass surgery, stroke, etc.), the oxygen demands of tissue can overwhelm the system, resulting in an oxygen deficit. The resulting ischemia can cause extensive and irreparable tissue damage. Further, reperfusion of ischemic tissue with oxygen is also known to cause considerable damage. For example, the production of mediators of the inflammatory cascade (e.g. cytokines and proteins involved in cell adhesion) can through many mechanisms cause extensive secondary damage on tissues and organs.

Historically, efforts to prevent or attenuate oxygen debt have consisted of increasing oxygen delivery. This method can be difficult or impossible to implement under many austere (e.g. rural, battlefield/prolonged extraction) conditions. Various temperature-mediated means have also been developed in an attempt to address these concerns in some situations. For example, it is a common practice to induce hypothermia in patients undergoing open heart surgery. However, these methods have certain drawbacks, such as expense and requiring significant time and skills. Thus, for the majority of patients or conditions in which reduction in oxygen consumption would be beneficial, production of hypothermia by known methods is not practical.

The prior art has thus far failed to provide methods to transiently and/or selectively inhibit oxygen consumption in tissues. Such a capability would be a particularly useful to treat or prevent injury due to ischemia and/or reperfusion.

### SUMMARY OF THE INVENTION

The present invention provides methods for modulating oxygen consumption and inflammation of an organism in order to prevent or lessen damage caused by ischemia and/or reperfusion. The method involves transient inhibition of protein synthesis or proteolysis in the organism by administration of agents that inhibit protein synthesis, and/or proteolysis. The transient inhibition may be global (decreasing protein synthesis in the entire organism) or localized (e.g. inhibiting proteins synthesis principally in one organ or region by administering the agents in a localized manner). The decrease in oxygen consumption that occurs as a result of administration of these agents induces mild to moderate hypothermia, and reduces the buildup of critical oxygen debt in the patient. Examples of suitable agents include antibiotics such as puromycin and anisomycin.

It is an object of this invention to provide a method of preventing or lessening tissue damage or inflammation due to ischemia or reperfusion or a combination thereof in a patient in need thereof. The method includes the step of administering to such a patient a sufficient amount of at least one agent that inhibits protein synthesis or proteolysis or a combination thereof. The agent is administered in an amount sufficient to temporarily decrease oxygen consumption needs in said patient. Alternatively or in addition, the agent is administered in an amount sufficient to reduce inflammation due to reperfusion. In particular, the agent is administered in an amount sufficient to inhibit protein production during reperfusion.

In one embodiment, the at least one agent inhibits protein synthesis, and in some cases may be an antibiotic such as puromycin or ansiomycin. In another embodiment, the at least one agent inhibits proteolysis.

The invention further provides a method of inducing hypothermia in a patient in need thereof. The method includes the step of administering to the patient a sufficient amount of at least one agent that inhibits protein synthesis or proteolysis or a combination thereof, thereby temporarily inducing hypothermia in the patient. In general, the method temporarily decreases the temperature of the patient by at least two degrees Celsius.

In one embodiment, the at least one agent inhibits protein synthesis, and in some cases may be an antibiotic such as puromycin or ansiomycin. In another embodiment, the at least one agent inhibits proteolysis.

The invention further provides a method of preventing or treating ischemia or reperfusion injury in a patient in need thereof. The method includes the step of administering to the patient a sufficient amount of at least one agent that inhibits protein synthesis or proteolysis, or a combination thereof. In one embodiment, the agent is administered in an amount sufficient to temporarily decrease oxygen consumption needs in the patient. In another embodiment, the agent is administered in an amount sufficient to reduce inflammation due to reperfusion. In yet another embodiment, the agent is administered in an amount sufficient to inhibit protein production during reperfusion.

In one embodiment, the at least one agent inhibits protein synthesis, and in some cases may be an antibiotic such as puromycin or ansiomycin. In another embodiment, the at least one agent inhibits proteolysis.

The invention further provides a method for modulating oxygen consumption in a patient. The method includes the step of administering to the patient a sufficient amount of at least one agent that inhibits protein synthesis or proteolysis or a combination thereof, thereby temporarily decreasing oxygen consumption needs in the patient.

In one embodiment, the at least one agent inhibits protein synthesis, and in some cases may be an antibiotic such as puromycin or ansiomycin. In another embodiment, the at least one agent inhibits proteolysis. The step of administering may induce hypothermia in the patient.

The invention further provides a method for controlling inflammation or production of inflammatory proteins in a patient in need thereof. The method includes the step of administering to the patient a sufficient amount of at least one agent that inhibits protein synthesis or proteolysis or a combination thereof, thereby controlling inflammation or production of inflammatory proteins in the patient.

In one embodiment, the at least one agent inhibits protein synthesis, and in some cases may be an antibiotic such as puromycin or ansiomycin. In another embodiment, the at least one agent inhibits proteolysis. The step of administering may induce hypothermia in said patient.

Further, in one embodiment, administration of the agent is carried out prior to a medical procedure. In general, the medical procedure is one that is known or anticipated to induce an inflammatory response, e.g. cardiopulmonary bypass.

### BRIEF DESCRIPTION OF THE DRAWINGS

FIGS. 1A and B. A, shows the individually-adjusted percentage change in VO₂ from baseline values for NS-C, DMSO-C and PSI rats approximately 1 hr from initial administration of protein inhibitors; B, shows the damped O₂ response of NS-C, DMSO-C and PSI rats and its association with a decline in core temperature.

FIG. 2A-D. ◯=Protein synthesis inhibitor (PSI); ♦=dimethylsulfoxide (DMSO) control. A, pO₂ (mm Hg) after time of administration of PSI; B, pCO₂ (mmHg) after time of administration of PSI; C, VO₂ (mL/kg/min) after time of administration of PSI; D, core temperature after time of administration of PSI.

FIG. 3A-D. Protein synthesis in various tissues after administration of A, puromycin, and B, anisomycin. Central venous O₂ saturation (%) after administration of C, puromycin, and D, anisomycin.

### DETAILED DESCRIPTION OF THE PREFERRED EMBODIMENTS OF THE INVENTION

The present invention provides pharmacological methods for modulating oxygen consumption in tissues. The method involves transient (temporary), global inhibition of protein synthesis, proteolysis, or both, in subjects that are under stress or may come under planned stress e.g. from oxygen deprivation, (ischemia) and/or susceptible to reperfusion injury, and that would benefit from a temporary decrease in oxygen consumption needs. Such a reduction in protein synthesis and/or proteolysis results in a temporary, global decrease in oxygen consumption in all major organ systems, making the organs more resistant to damage from decreases in oxygen delivery, and reducing the degree to which oxygen delivery must be reestablished to support the organ system. The methods result in a temporary but significant reduction in the oxygen utilization needs of the subject, and also result in mild to moderate hypothermia in the subject. Inhibition of protein synthesis also reduces the degree of reperfusion injury. Without being bound by theory, it is believed that this occurs by 1) reducing the magnitude of tissue ischemia and subsequent reperfusion injury based on the length and severity of ischemia; and 2) reducing or preventing the production of protein mediators of the inflammatory cascade (such as cytokines and proteins involved in cell adhesion) when oxygen delivery is reestablished after ischemia. Thus, by practicing the methods of the present invention, the initial amount of damage from ischemia is reduced, and tissue and organ damage resulting after reestablishment of oxygenation (reperfusion) is also attenuated, thereby reducing the incidence of secondary tissue damage and multi-system organ failure.

The methods of the present invention involve the administration of inhibitors of protein synthesis, and/or proteolysis, or both, to a patient in need thereof. The protein synthesis inhibitors may inhibit protein synthesis at any stage of the process, e.g. initiation, translation, post-translational processing, etc. Those of skill in the art will recognize that many agents exist that, when administered to a subject, cause inhibition of protein synthesis. Examples include but are not limited to: antibiotics (e.g. puromycin, anisomycin, doxycline, tetracycline, cycloheximade); ribozymes; small inhibitory RNA; plant extracts (e.g. emetine); flavonoids (e.g. genestine); toxins (e.g. ricin); antisense oligonucleotides (for example, those that bind to mRNA encoding proteins of interest); etc. Any agent that inhibits protein synthesis at any stage and by any mechanism may be utilized in the practice of the present invention, so long as the agent is administered in a quantity and form that are not toxic to the subject.

In one aspect of the invention, a new use for known protein synthesis and/or proteolysis inhibitors (e.g. antibiotics) is provided. Agents such as antibiotics are traditionally used to inhibit protein synthesis in pathogenic prokaryotic species. According to the methods of the present invention, such agents are used to inhibit protein synthesis (temporarily) in eukaryotic organisms, such as humans.

The methods of the present invention further comprise administering inhibitors of proteolysis as a means to reduce oxygen consumption. Inhibition of proteolysis may reduce oxygen consumption directly, and/or indirectly by inhibiting protein synthesis, for example, in the case of proteins whose synthesis is controlled by feedback inhibition. In addition, proteolysis requires energy and thus its inhibition will reduce oxygen consumption.

Those of skill in the art will recognize that many agents exist which can be used to inhibit proteolysis in a patient in need thereof. Examples of such agents include but are not limited to tissue inhibitors of metalloproteinases and plant extracts such as emetine.

The methods of the present invention involve the transient or temporary reduction of oxygen consumption needs of an organism. By “transient” or “temporary” we mean that the effect produced by administration of the agents is not permanent, but rather lasts for a relatively short period of time. In general, according to the methods of the invention, the reduction of oxygen consumption that is induced persists for about one to about six hours, and preferably for about one to about two hours, depending on the agent(s) used. Further, the optimal decrease in oxygen consumption will be in the range of about 10% to about 50%, and preferably in the range of about 20% to about 50% compared to suitable controls. Those of skill in the art are well-acquainted with the concept of establishing appropriate controls for such comparisons. Those of skill in the art will recognize that the period and degree of reduced oxygen consumption may be altered by manipulation of parameters such as the type(s) of agent(s) used, the amount(s) of agent(s) administered, by repeated administration of agents, the route of admonistration, and the circulatory status of the organism or tissue(s) of interest at the time of administration, and the like. The methods may thus be tailored to specific needs of the situation, e.g. during transport of a patient from a remote site to a suitable treatment facility, or while other beneficial procedures (e.g. diagnostics, surgical procedures, etc.) are being performed it may be beneficial to induce longer periods of reduced oxygen consumption.

The methods of the present invention result in global inhibition of protein synthesis and/or proteolysis in an organism. By “global inhibition” we mean that most major organs or organ systems of the organism will exhibit a decrease in protein synthesis activity (and/or proteolysis) as a result of the implementation of the methods of the invention. In general, the decrease will occur in all major organs and organ systems including but not limited to liver, kidney, heart, lung, digestive system, muscles, brain, etc. Further, the decrease will be in the range of from about 50% to about 90%, and preferably in the range of from about 80% to about 90%, compared to suitable control organisms. Those of skill in the art are well-acquainted with the concept of establishing appropriate controls for such comparisons.

Those of skill in the art will recognize that the amount of agent or compound to be administered according to the methods of the present invention will vary from agent to agent, and from subject to subject, and is best determined by a skilled practitioner such as a physician. Similarly, the method of administering the agent will vary according to several parameters, including but not limited to: the agent(s) being administered; the circumstances surrounding administration (e.g. operating room vs battlefield); the gender, age, overall health, extent of injury etc. of the patient; etc. In general, the agent or compound may be in the form of a solid such as a pill or tablet, as a liquid or emulsion, e.g. for IV or IM administration, etc. In general, administration may be oral, intravenous, intramuscular, via inhalation, intraperitoneally, intrathecally, or by any other suitable means.

The agents that are administered in the practice of the present invention may be administered either alone, or as a combination of two or more agents, i.e. in a “cocktail” of agents, each of which inhibits protein synthesis and/or proteolysis. In one embodiment, the agents are administered as a cocktail of puromycin and anisomycin. In addition, the practice of the methods of the present invention can be coupled with other suitable techniques that are known to those of skill in the art, depending on the condition being treated, such as temperature-induced hypothermia, various methods to staunch blood flow, administration of other beneficial medicaments, surgical procedures, medical procedures, etc.

Those of skill in the art will recognize that many conditions exist for which the practice of the methods of the present invention could be usefully applied. Examples include but are not limited to hemorrhagic and traumatic shock (such as those which result in accidents or in a battlefield situation, or in complex surgeries or medical conditions), sepsis and septic shock, cardiogenic shock, cardiopulmonary bypass surgery, myocardial ischemia/infarctions, stroke, traumatic brain injury, organ transplantation, and any other situation in hich globabl or isolated organ injury is expected to occur, etc. The therapeutic benefits of the methods of the present invention may be applied in any setting in which, for example, ischemia or inflammation may play a role in tissue damage. The methods are useful in preventing primary tissue ischemia or dysoxia from various insults ranging from those that affect the whole body (hemorrhage, sepsis or cardiogenic shock), to those that affect individual organs such as traumatic brain injury, myocardial infarction, stroke and others. The reduction in inflammation caused by the practice of the present invention may be useful in such procedures as cardiopulmonary bypass and post-transplant organ injury, as well as in extending the life of organs to be transplanted, or in situations in which the insult(s) produce a primary inflammatory response to an organ or system such as burns, large wounds or long bone fractures, acute lung injury, etc.

The present invention also provides methods for transiently inducing hypothermia in a patient or organism in need thereof, and preferably for inducing mild to moderate hypothermia.

By “mild to moderate hypothermia” we mean that the core temperature of the organism is between 27 and 35 degrees Celsius. The method thus involves inducing a reduction in body temperature of the patient by administering agents that inhibit protein synthesis and/or proteolysis. In general, according to the methods of the invention, the body temperature of an individual so treated will decrease in the range of from about 2 to about 10 degrees C., and preferably from about 3 to about 7 degrees C. Further, the reduction in temperature will be maintained for a period of time ranging from about 1 to about 6 hours, and preferably for from about 2 to 4 hours.

The present invention provides methods for preventing or lessening tissue damage due to ischemia or reperfusion in a patient in need thereof. Alternatively, the invention provides a method of treating ischemia or reperfusion injury in a patient in need thereof. In yet another embodiment, the invention provides a method for modulating oxygen consumption in a patient.

The methods of the present invention involve administering to the patient a sufficient amount of at least one agent that inhibits protein synthesis or proteolysis, or both. The result of such administration is that the oxygen consumption needs of the patient are temporarily decreased, thereby preventing or lessening damage and injury due to ischemia and/or reperfusion. The methods may be carried out prophylactically (i.e. before and in anticipation of a need for decreased oxygen consumption, such as prior to a surgical procedure where ischemia and/or reperfusion is/are likely to occur) in order to prevent or lessen the impact of the ischemia and/or reperfusion. Alternatively, the methods may be carried out during or after the occurrence of ischemia and/or reperfusion in order to prevent or lessen damage or injury to tissues and organ systems that might occur as a result of ischemia and/or reperfusion. Those of skill in the art will recognize that in general, the sooner the methods of the present invention can be implemented after onset of ischemia and/or reperfusion, the more successful the treatment is likely to be. Preferably, administration of agents to reduce oxygen consumption will be administered within 0-60 minutes post-onset, and preferably within about 0 to 20 minutes, and most preferably within about 0-10 minutes or less after onset of ischemia and/or reperfusion.

While in some embodiments of the invention, the combined deleterious effects of ischemia and reperfusion are treated, the methods of the present invention may also be used to treat reperfusion injuries without treating ischemia. This may occur, for example, in the case where it is not possible to reach and treat a patient in time to treat ischemia, or in cases where ischemia does not occur or is treated in some other manner, but in which the patient still runs the risk of substantial tissue damage due to inflammation, e.g. as a result of reperfusion, or from other causes. For example, some injuries such as burn and trauma result in massive tissue injury which are capable of invoking the upregulation and production of proteins involved in producing inflammatory reactions. These reactions when severe enough can result in sepsis, organ dysfunction, and death. In such cases, administration of agents that inhibit protein synthesis and/or proteolysis to temporarily disrupt this mechanism may provide substantial benefits by inhibiting tissue damage associated with the production of noxious, injurious proteins, especially those associated with the inflammatory response (e.g. cytokines).

The use of protein synthesis inhibition to reduce organ damage might also be applied to selective organ systems through special delivery strategies. For example, patients with significant pneumonia or other acute lung injury in which inflammation is localized could inhale an aerosol of protein synthesis inhibitors thus reducing total systemic delivery and focusing the effects of the inhibitors on lung tissue. Other examples might include delivery of protein synthesis inhibitors to the ischemic heart during angioplasty or the ischemic brain during interventional radiological procedures. The use of interventional radiology to open and stent vessels to ischemic organ systems is increasing thus allowing for the potential of selective delivery of these protein synthesis inhibitors to target organs. Within the field of neurosurgery, catheters are now placed directly from the skull into portions of the brain for monitoring and sampling of biologic processes. Protein synthesis inhibitors could be selectively injected into these areas to reduce injury from a wide variety of insults such as traumatic brain injury, massive stroke, brain edema, or intracranial hemorrhage from other diseases. Delivery of these agent might be in bolus form or continuous infusion.

The use of protein synthesis inhibitors to enhance the process of organ transplantation could also be envisioned by perfusing the harvested organ either in vivo or ex vivo prior to its transplant. This could enhance the window of opportunity to transplant the organ by decreasing ischemia and preventing the production of injurious proteins and thus inflammation upon reperfusion.

### EXAMPLES

### Example 1

### Changes in VO₂ and Core Temperature Following Administration of Protein Synthesis Inhibitors

The ability of transient global inhibition of protein synthesis to reduce total cellular metabolism, and thus increase the tolerance of the body to acute reductions in tissue oxygen delivery, as occurs in traumatic or hemorrhagic shock, was examined. The desired end results of such investigations are to extend the window for post-trauma salvage, and reduce the probability of post-resuscitation complications, such as multi-system organ dysfunction (MODS).

The effects of a combination of two protein synthesis inhibitors (PSI), anisomycin and puromycin, on whole-body oxygen consumption (O₂) of conscious adult male rats had previously been investigated, and it had been demonstrated that this particular cocktail contributes to statistically significant, albeit clinically modest, reduction in O₂ relative to controls.

To determine the physiological modulatory potential of an agent, resting metabolic rate (O₂), core temperature, and hemodynamic parameters were monitored in conscious animals surgically implanted with intravascular catheters and an intraperitoneal temperature transmitter. Implantation of these devices allowed both intravenous administration of experimental drugs and continuous physiological monitoring of the animal simultaneously with O₂ measurements without handling or removing the animal from the chamber. Animals were surgically implanted under surgically sterile conditions with venous and arterial catheters and a temperature transmitter. The right femoral artery and right external jugular vein were isolated and cannulated with PE-50 catheters; these had been treated prior to implantation with TD-AC heparin to reduce clotting. The transmitter (E-Mitter; VitalView® Data Acquisition System; Minimitter, Inc. OR) was implanted in the peritoneal cavity. Catheters were exteriorized at the animal's nape and attached to a stainless-steel swivel and tether (Instech Laboratories); the tether device protects the catheters from damage and interference from the animal. Swivel holders were mounted on the top of the cage with a spring counterbalance lever arm that protected the catheter from tangling and allowed the animal unrestricted movement throughout the cage and free access to food and water. Animals were allowed to recover for a minimum of 1 week before monitoring, when they had regained preoperative weight.

Oxygen uptake and carbon dioxide production were measured simultaneously in an open-circuit flow-through system. The metabolic chamber was a Plexiglas cylindrical chamber (28×11.5 cm; volume 1200 cm³). Excurrent gas flow was monitored by a flowmeter (TSI 4100; TSI Incorporated, MN), and flow rates adjusted between 900-1200 mL/min to maintain [CO₂]<1% to minimize hypercapnia, and to avoid spurious elevation of metabolic rates from evaporative water loss accumulation. Excurrent air was passed through a small drying column (Drierite) before entering the subsampling stream to the analyzers. Each animal was weighed, and sealed in the chamber, and the chamber was immersed in a water bath to maintain a stable chamber temperature (22.4±0.6° C.). Concentrations of O₂ and CO₂ were sampled from incurrent and excurrent air streams, and measured with BIOPAC MP150 systems DA100 C (BIOPAC Systems, CA). The analyzers were calibrated daily with commercial gas preparations. Rates of oxygen consumption O₂ were calculated by subtracting the fractional concentration of O₂ in the outflow gas from that in the inflow gas and multiplying by the excurrent flow rate. Rates of carbon dioxide consumption CO₂ was calculated by multiplying the gas flow and the CO₂ concentration in the outflow air stream. Barometric pressure and chamber temperature were used to convert measures to STPD.

Animals were assigned randomly to one of 3 treatments, and treatment order was randomized by day. Animals were measured for at least 100 minutes to establish resting baseline metabolic rates. Subsequently, rats were injected with 1 of the 3 treatment preparations. Saline control rats (NS-C) were given a single IV bolus of 2 mL sterile normal saline (0.9%). The DMSO carrier control consisted of a 2 mL IV bolus of pH-adjusted carrier DMSO administered 5% by volume in phosphate buffered saline (DMSO-C). The protein synthesis inhibitor treatment was a cocktail of puromycin at a dose of 20 mg/kg and anisomycin at 100 mg/kg (Sigma-Aldrich Chemicals) dissolved in mass-adjusted volume of 5% DMSO-phosphate buffered saline. For all solutions, pH was adjusted to 7.0-7.1 with 0.1 N NaOH or 0.1 N HCl, and warmed to 37° C. before injection; all solutions were administered via the jugular vein through the implanted catheter. Metabolic measurements were then continued for another 120 minutes. If the arterial line remained patent, arterial blood was sampled at baseline and at designated intervals (15, 30, 45, 60 and 120 min post-injection) for arterial blood gases, electrolyte, and lactate status using an ABL blood gas machine (Radiometer, Copenhagen). At the end of the experiment, animals were euthanized by injection of 0.1 mL pentobarbital through the jugular catheter.

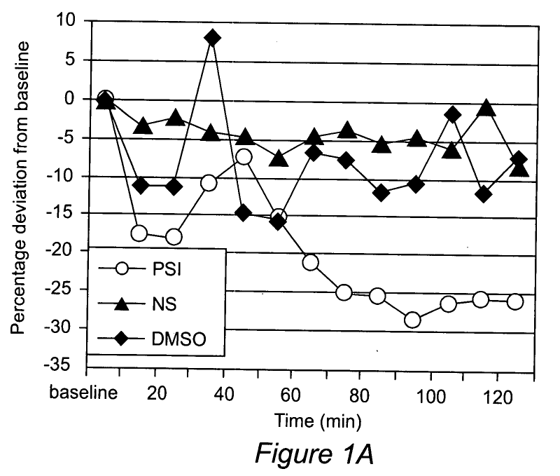

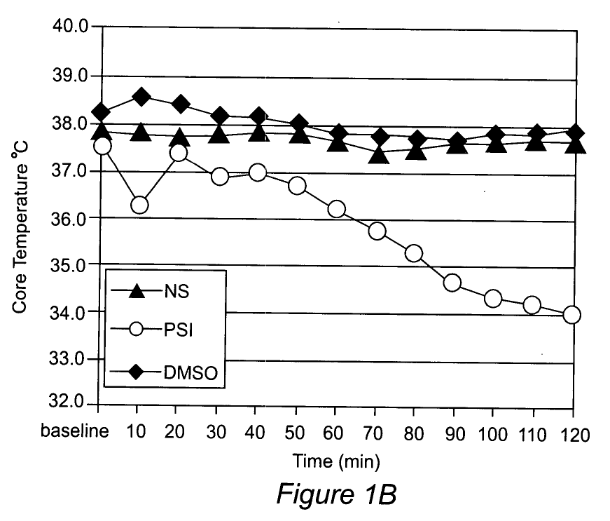

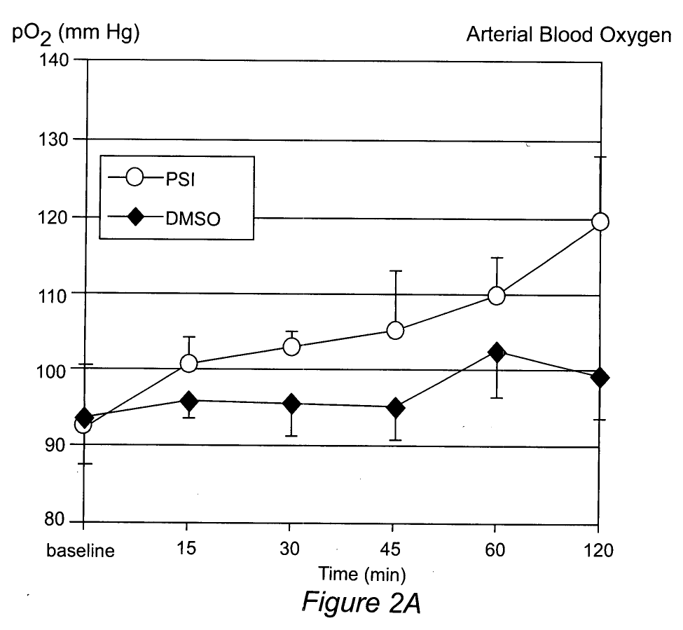

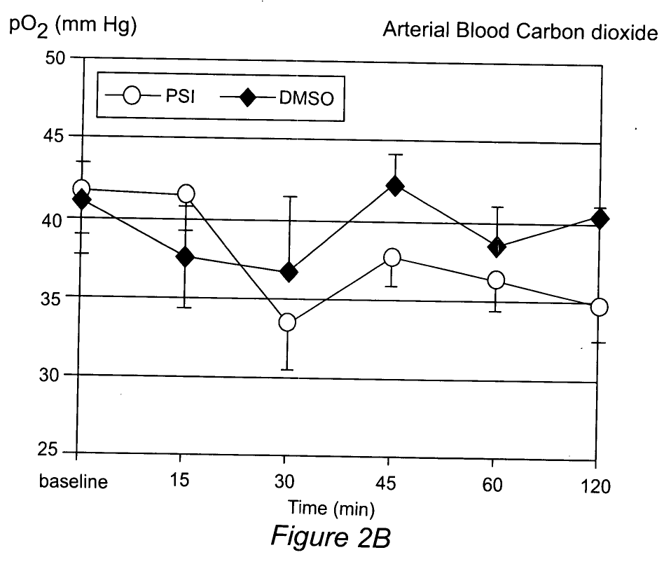

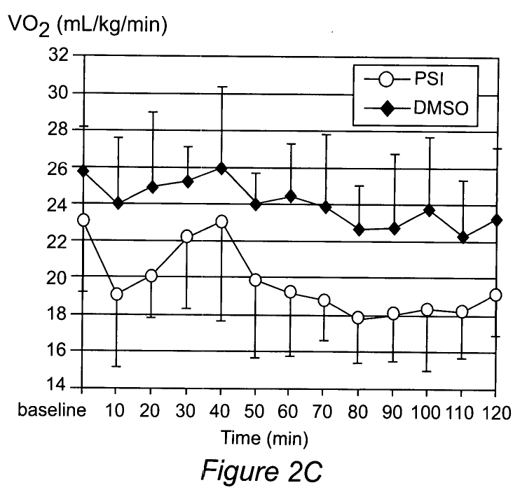

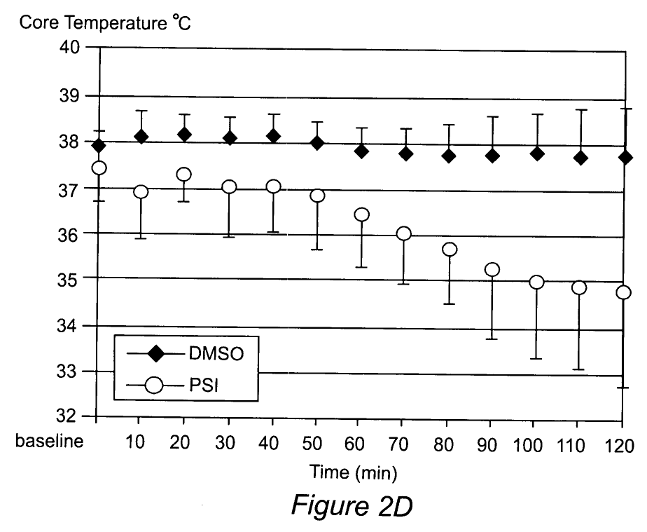

Metabolic rate data were converted to units of mL kg⁻¹ min⁻¹. Procedural controls and experimental treatments were compared with a single-factor repeated-measures analysis of covariance, with baseline values as covariate (RM-ANCOVA). All data are presented in FIGS. 1 and 2 as means±standard deviations (SD). FIG. 1A shows the individually-adjusted percentage change in VO₂ from baseline values. As can be seen, both NS-C (n=3) and DMSO-C rats (n=3) showed almost no change in O₂ (−8% change), whereas PSI rats exhibited a 26% decline from baseline approximately 1 hr from initial administration.

FIG. 1B indicates that the damped O₂ response of PSI rats is associated with a 3.51° C.±1.87° C. decline in core temperature. In contrast, core temperatures of NS and DMSO control rats did not vary appreciably from baseline (−0.03±0.93° C. and 0.27±1.37° C. respectively).

In addition, complete blood gas data sets have been obtained for the animals, and all values were within published normal values for Sprague-Dawley rats (Baker, Lindsey and Weisbroth 1979. The laboratory rat. Vol. I. Biology and diseases). There were no discernable effects of treatment on values; all rats showed lactate levels <2.

These results demonstrate that administration of inhibitors of protein synthesis significantly reduce oxygen consumption and core body temperature without apparent harm to the animals.

### Example 2

### Metabolic Down-Regulation by Protein Synthesis Inhibition Agents in a Conscious Rat Model

The single major cause of combat death is hemorrhage; however it has been estimated that approximately 20% of battlefield casualties are potentially salvageable. The major differences between outcomes for victims of injury-induced hemorrhage in combat, as opposed to the civilian, setting are time to definitive care and resource availability. The “golden hour” for definitive care is not feasible on the battlefield because of prolonged evacuation times and constraints on resuscitation supplies (e.g. fluids, blood, and oxygen). Therefore it is critical to develop therapies that will extend the window of opportunity for the injured warfighter to survive life-threatening hemorrhage.

The hemorrhagic shock state entails a mismatch between cellular oxygen delivery and demand. Historically, shock has been viewed as being primarily a problem with oxygen delivery; therefore therapeutic strategies typically involve the use of high-flow oxygen, volume expanders, and blood replacement. In contrast, the present invention provides an alternative strategy, namely that reduction of total cellular metabolism will reduce somatic oxygen demand, and thus increase the tolerance of the body to acute reductions in tissue oxygen delivery. Because protein synthesis accounts for ˜35% of total cellular energy demands, this objective may be achieved via the mechanism of a transient global inhibition of protein synthesis.

The modulation of whole-body oxygen demand in a conscious rat model by a combination of pharmaceutical agents known to induce transient inhibition of protein synthesis was inestigated. The performance of a cocktail of the antibiotics puromycin and anisomycin in comparison to carrier controls on whole body oxygen consumption, oxygen debt, core body temperature, and hemodynamic stability, was compared. Anisomycin is derived from Streptomyces griseolus; it inhibits translation by binding to 60S ribosomal subunits and blocking peptide bond formation, thereby preventing elongation and causing polysome stabilization. Puromycin is an aminonucleoside antibiotic produced by S. alboniger; it specifically inhibits peptidal transfer and causes premature termination by acting as a structural analogue of an aminoacyl-tRNA. Transient global inhibition of protein synthesis reduces total cellular metabolism, therefore reducing whole body oxygen consumption, and thus the buildup of critical oxygen debt.

Methods: Male Sprague-Dawley rats 300-390 g were chronically instrumented under surgically sterile conditions and allowed to recover for 7-10 d. Animals were assigned randomly to one of 2 treatments: DMSO carrier control (DMSO)=2 mL IV bolus of pH-adjusted carrier DMSO (5 vol %) in phosphate buffered saline PBS (n=7); or Protein synthesis inhibitor (PSI)=puromycin (20 mg/kg)+anisomycin (100 mg/kg) in mass-adjusted volume of 5% DMSO-PBS; delivery volume ˜2 mL (n=7). VO₂, CO₂, T_{b}, and hemodynamic variables were measured simultaneously in an open-circuit flow-through system. Arterial blood gases were measured every 15 min. Data were analyzed by repeated measures ANOVA adjusted for baseline, and simple contrasts. Results are presented as means±SD (indicated by dashed lines).

Mean pO₂ levels of PSI rats (◯) increased significantly over time (p=0.02) and were higher at 2 2 hr (p=0.06) than that of DMSO controls (♦) (FIG. 2A). Mean pCO₂ levels of PSI rats at 2 hrs were significantly reduced from baseline levels (p=0.009), and were lower than those of DMSO rats at 2 hrs (p=0.007) (FIG. 2B); mean pCO₂ of DMSO rats at 2 hrs did not differ significantly from baseline (p=0.862).

Oxygen consumption—Baseline VO₂ did not differ between DMSO and PSI rats. As seen in FIG. 2C, PSI rats showed a statistically significant overall reduction in VO₂ compared with DMSO rats (treatment F_{1,11}=9.549, p=0.010). DMSO rats showed a non-significant drop in VO₂ over 2 hrs (4.4% change). PSI rats exhibited a 22.8% decline from baseline at 1 hr from initial administration. An initial 25% drop in VO₂ occurred 10 min after administration, followed by an increase of VO₂ to resting levels at 30 and 40 min then a subsequent decline.

Core temperature—Baseline T_{body} did not differ between DMSO and PSI rats. As seen in FIG. 2D, PSI rats showed a statistically significant overall reduction in T_{body} compared with DMSO rats (treatment F_{1,11}=5.791, p=0.037). For PSI rats, note the transient plateauing of T_{body} concomitant with the increase in VO₂ between 20-40 min post-injection.

Hemoglobin levels were similar for both groups (13.2±SD 0.8 g/dL; p=0.621). Hb saturation levels sO₂ did not differ (93.3±SD 0.6%; p=0.725).

Conclusions: Hemorrhagic shock is associated with ischemic metabolic insufficiency caused by inadequate cellular perfusion. Decreased perfusion results in impaired cellular oxygen delivery; if prolonged, cells become irreversibly damaged and are more susceptible to lethal reperfusion injury. These data suggest that a therapeutic model predicated on reduction of tissue oxygen demand rather than an augmentation of oxygen delivery will be of promise in the development of unconventional but practical resuscitation therapies in the combat and other arenas.

### Example 3

### Down-Regulation of Protein Synthesis in Rat Model

Methods: Sprague Dawley rats (300-350 grams body weight) were anesthetized with Saffan (a cocktail of two steroid anesthetics (Alfaxalone and Alfadolone Acetate). Polyethylene catheters were implanted in the carotid artery (determination of mean arterial blood pressure, heart rate and arterial oxygen saturation), femoral vein (infusions), jugular vein (determination of central venous pressure and central venous oxygen saturation) and trachea (facilitate breathing). Following surgical recovery, baseline data was obtained. The animals then received either vehicle (5% dimethyl sulfoxide in phosphate buffered saline) or a protein synthesis inhibitor intravenously over a period of 1-2 minutes. Thirty minutes following administration of the PSI or vehicle, all animals received ³⁵S-methionine (750 μg/kg, i.v. bolus). Following thirty minutes of label uptake, all animals are placed on a ventilator and administered cold (5° C.) saline to stop remaining protein synthesis and wash out unincorporated ³⁵S-methionine remaining in the blood. Following sacrifice, multiple tissues (liver, kidney, heart, lung, ileum, duodenum, muscle, cerebellum and cerebrum) were removed and immediately frozen. Tissues were thawed, homogenized and washed. The supernatants were analyzed in a standard beta scintillation counter, and the results were referenced to protein content.

Results: Genistein was a poor inhibitor of protein synthesis, and caused toxicity at low doses.

Doxycycline also provided minimal inhibition of protein synthesis at doses (<⅓ of LD₅₀) causing high (˜66%) mortality. However, puromycin and anisomycin both resulted in substantial inhibition of protein synthesis.

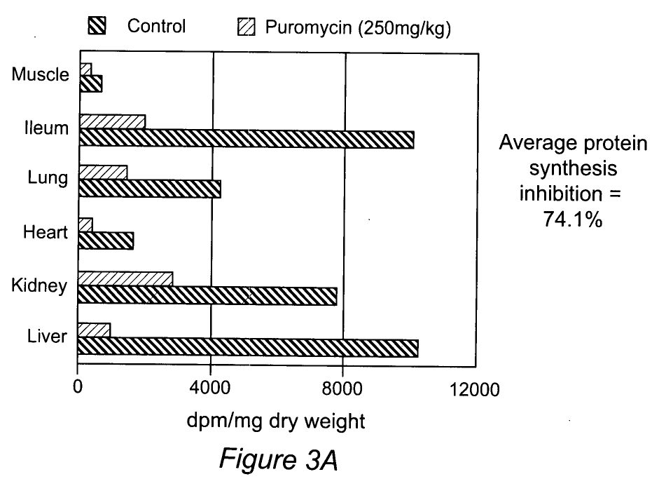

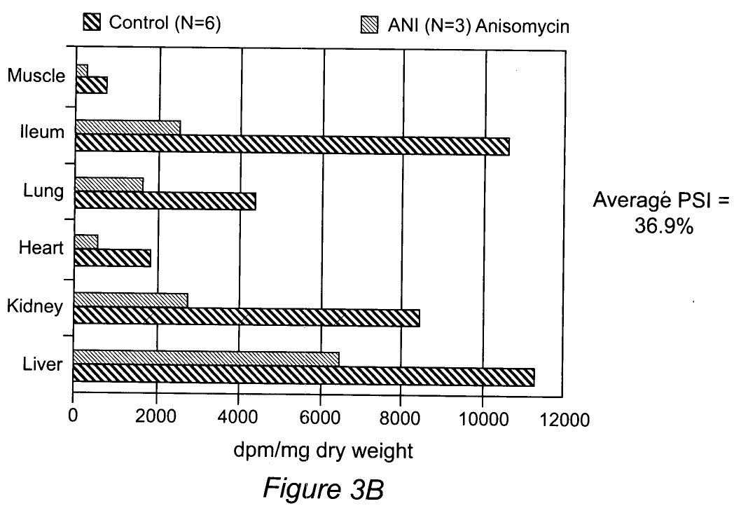

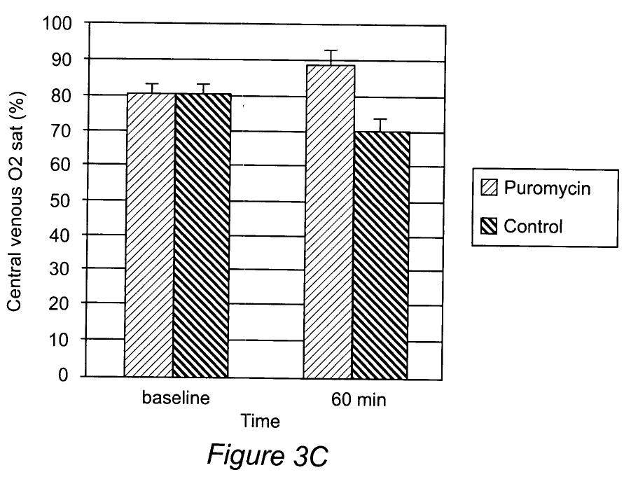

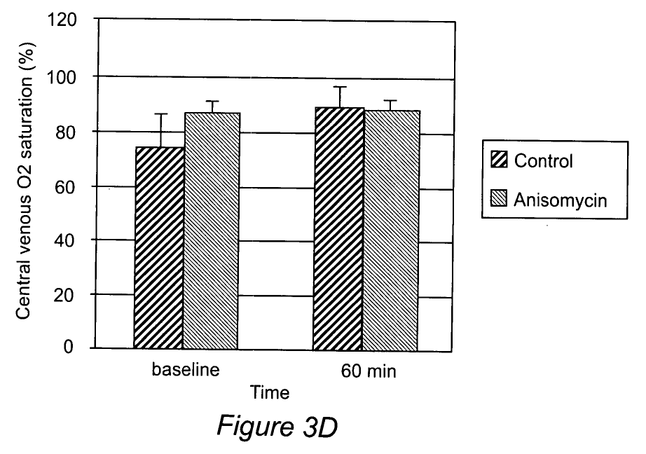

Puromycin was chosen because it acts to block protein synthesis at the initiation step. Specifically, it acts as an analog of the 3′ terminal end of aminoacyl-tRNA, causing premature chain termination. Protein synthesis inhibition and central venous saturations resulting from administartion of puromycin are shown in FIGS. 3A and B, respectively.

Anisomycin acts at a later step, inhibiting translation by blocking formation of the 80S ribosomal complex from 60S subunits. A combination of these inhibitors acted synergistically, resulting in an average inhibition of 81 percent, as illustrated in Table 1.

TABLE 1
Percent Inhibition of Protein Synthesis (compared to mean control
values) in various tissues one hour following intravenous administration
of a puromycin (100 mg/kg)/anisomycin (20 mg/kg) cocktail.

| | Percent PSI (vs. |
|---|---|
| Tissue | Controls): |
| Liver | 79.1 |
| Kidney | 75.4 |
| Heart | 79.9 |
| Lung | 86.2 |
| Ileum | 73 |
| Duod. | 68.4 |
| Muscle | 85.5 |
| Cerebellum | 86.7 |
| Cerebrum | 94.2 |
| Overall | 81 |

The central venous saturation of these animals also increased one hour following administration of the cocktail from 70 to 77%. In the absence of changes in cardiac output or arterial oxygen content, this would represent a decrease in VO₂ of at least 10%. However, this represents venous blood sampled from the superior vena cava, which drains a small amount of tissue. A truly mixed venous sample from the pulmonary artery would likely have demonstrated an even larger increase in oxygen saturation, implying a larger drop in oxygen consumption.

This example demonstrates that administration of certain inhibitors of protein synthesis rapidly and safely reduce protein synthesis in all major organs by a significant degree in a clinically relevant time period.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method of preventing or lessening tissue damage or inflammation due to ischemia or reperfusion or a combination thereof in a patient in need thereof, comprising the step of
administering to said patient a sufficient amount of at least one agent that inhibits protein synthesis or proteolysis or a combination thereof.

2. The method of claim 1, wherein said agent is administered in an amount sufficient to temporarily decrease oxygen consumption needs in said patient.

3. The method of claim 1, wherein said agent is administered in an amount sufficient to reduce inflammation due to reperfusion.

4. The method of claim 1, wherein said agent is administered in an amount sufficient to inhibit injurious protein production during reperfusion.

5. The method of claim 1, wherein said at least one agent inhibits protein synthesis.

6. The method of claim 1, wherein said at least one agent is an antibiotic.

7. The method of claim 6, wherein said antibiotic is selected from the group consisting of puromycin and ansiomycin.

8. The method of claim 1, wherein said at least one agent inhibits proteolysis.

9. A method of inducing hypothermia in a patient in need thereof, comprising the step of
administering to said patient a sufficient amount of at least one agent that inhibits protein synthesis or proteolysis or a combination thereof, and thereby temporarily inducing hypothermia in said patient.

10. The method of claim 9, wherein a body temperature of said patient temporarily decreases by at least two degrees Celsius.

11. The method of claim 9, wherein said at least one agent inhibits protein synthesis.

12. The method of claim 11, wherein said at least one agent is an antibiotic.

13. The method of claim 12, wherein said antibiotic is selected from the group consisting of puromycin and ansiomycin.

14. The method of claim 9, wherein said at least one agent inhibits proteolysis.

15. A method of preventing or treating ischemia or reperfusion injury in a patient in need thereof, comprising the step of
administering to said patient a sufficient amount of at least one agent that inhibits protein synthesis or proteolysis or a combination thereof.

16. The method of claim 15, wherein said agent is administered in an amount sufficient to temporarily decrease oxygen consumption needs in said patient.

17. The method of claim 15, wherein said agent is administered in an amount sufficient to reduce inflammation due to reperfusion.

18. The method of claim 15, wherein said agent is administered in an amount sufficient to inhibit injurious protein production during reperfusion.

19. The method of claim 15, wherein said at least one agent inhibits protein synthesis.

20. The method of claim 19, wherein said at least one agent is an antibiotic.

21. The method of claim 20, wherein said antibiotic is selected from the group consisting of puromycin and ansiomycin.

22. The method of claim 15, wherein said at least one agent inhibits proteolysis.

23. A method for modulating oxygen consumption in a patient, comprising the step of
administering to said patient a sufficient amount of at least one agent that inhibits protein synthesis or proteolysis or a combination thereof, and thereby temporarily decreasing oxygen consumption needs in said patient.

24. The method of claim 23, wherein said at least one agent inhibits protein synthesis.

25. The method of claim 24, wherein said at least one agent is an antibiotic.

26. The method of claim 25, wherein said antibiotic is selected from the group consisting of puromycin and ansiomycin.

27. The method of claim 23, wherein said at least one agent inhibits proteolysis.

28. The method of claim 23, wherein said step of administering induces hypothermia in said patient.

29. A method for controlling inflammation or production of inflammatory proteins in a patient in need thereof, comprising the step of
administering to said patient a sufficient amount of at least one agent that inhibits protein synthesis or proteolysis or a combination thereof, and thereby controlling inflammation or production of inflammatory proteins in said patient.

30. The method of claim 29, wherein said at least one agent inhibits protein synthesis.

31. The method of claim 30, wherein said at least one agent is an antibiotic.

32. The method of claim 31, wherein said antibiotic is selected from the group consisting of puromycin and ansiomycin.

33. The method of claim 29, wherein said at least one agent inhibits proteolysis.

34. The method of claim 29, wherein said step of administering induces hypothermia in said patient.

35. The method of claim 29, wherein said step of administering is carried out prior to a medical procedure.
